(19) United States
(12) Patent Application Publication
(10) Pub. No.: US 2025/0102700 A1
(43) Pub. Date: Mar. 27, 2025

(54) COMPUTER-IMPLEMENTED METHOD FOR MULTI-SCALE UNSUPERVISED CLASSIFICATION OF ELECTRO-FACIES

(71) Applicant: PETRÓLEO BRASILEIRO S.A. – PETROBRAS, Rio de Janeiro (BR)

(72) Inventor: PEDRO PAULO PIRES DE DEUS ROCHA, Rio de Janeiro (BR)

(73) Assignee: PETRÓLEO BRASILEIRO S.A. – PETROBRAS, Rio de Janeiro (BR)

(21) Appl. No.: 18/776,036

(22) Filed: Jul. 17, 2024

(30) Foreign Application Priority Data
Sep. 21, 2023 (BR) 1020230192440

(51) Int. Cl. G01V 20/00; G06F 30/28; G16C 60/00

(52) U.S. Cl. CPC G01V 20/00; G06F 30/28; G16C 60/00

(57) ABSTRACT

The present invention relates to a machine learning unsupervised method to improve results obtained in electro-facies models. In this method it is possible to subdivide profile data into different unsupervised classes interactively and intuitively based on rock data information, seeking not only to meet the need to respect profile data values, but also to represent the geological knowledge that exists in the labeled data which serves as a guide in decision making to define which classes should be subdivided or attached. In the method, the result is constructed little by little and intuitively, just like the process of analyzing an outcrop or core. Initially, profile data is used to identify the most relevant and easily separable macro characteristics, such as reservoir and non-reservoir rock. Additionally, there is the possibility of interactively subdividing or attaching classes to test scenarios and quickly validate concepts. In this sense, it is quite intuitive to start from a macro analysis and gradually identify more specific characteristics based on the geological knowledge of a specialist or what the rock data indicates, so that the method allows countless interactions to be carried out until reaching the desired result.

### FIELD OF THE INVENTION

The present invention applies to the industrial plant of the oil, natural gas and energy industry. Preferably, the present invention falls within the technical field of modeling, simulation and evaluation of reservoirs.

### BACKGROUND OF THE INVENTION

The term Electro-facies was first defined by Serra & Abbot (1980) referring to an approximation of lithofacies when reading geophysical well profiles. In other words, electro-facies can be considered as a product of statistical classification and cluster of well data (profile and rock) with the aim of characterizing and individualizing patterns with geological meanings. However, it is observed that this characterization is not trivial, since the profiles do not have the same geological dimension, and two points that are close in the profile dimension are not necessarily close in the geological dimension. Therefore, different techniques can be used to define electro-facies models, such as discriminant analysis, multivariate regression, neural networks, cut-offs, visual interpretation of patterns in profiles, etc.

The input data for electro-facies models are electrical profiles and, usually, rock data. Both data are provided from structured tables in which the depth of each variable sample is arranged in the rows of the table and the variable values or rock labels are arranged in the columns. It is important that the electrical profiles used in the construction of the model are capable of characterizing the different classes of rock that the model aims to predict. Therefore, there is a need for coherence between the rock classes and the available electrical profile data. Due to several factors, this coherence cannot always be achieved and, consequently, the generated model is unable to predict certain classes with an adequate level of uncertainty.

In this context, the extensive use of supervised machine learning techniques in electro-facies classification tasks finds that the main difficulty lies in the consistent integration between rock data and profile data and not in the classification method. Among the several factors that contribute to this difficulty, two are extremely relevant: the first factor is that the classification of the rock data used as a label for these methods is subjective and, depending on the purpose, the granularity of the classification can completely change the label. In other words, the level of detail that a given rock can receive in its labeling is directly linked to the objective for which the label is being made. The second factor is the issue of the sample representation we have of rock types, which, unfortunately, is something that can hardly be improved. Electro-facies studies mostly have less than 1% of labeled data (rock classes where several rock samples are analyzed and given a name) and, when it comes to supervised machine learning, this amount of label is very low. This implies that the resulting models have a strong bias towards some classes and do not always represent all classes that occur in the context.

In this scenario, unsupervised machine learning methods become more attractive alternatives. One of the great advantages of these types of methods is the possibility of working with the complete data (wherein the complete data is a table where the lines represent the depth at which each sample was acquired, whether electrical profile or rock, and the columns are the values of each property, where for the electrical profile they are values, and for rocks they are names), without being limited only to the labeled parts. This allows all combinations of profile responses in the range of interest to be considered. However, there are disadvantages inherent to almost all unsupervised methods, such as the need to impose the number of classes into which the algorithm will divide the data, as well as the need for geological knowledge to intuitively search for the meaning of the clusters.

In this sense, an interactive multi-scale unsupervised classification method was developed seeking to improve the results obtained in electro-facies models. It is possible to subdivide the profile data into different unsupervised classes interactively and intuitively based on rock data information. Unsupervised methods use the concept of data driven to the extreme, as there is no interpretation imposed on the classification process. In this sense, the proposed methodology seeks not only to meet the need to respect the values of profile data, but also to represent the geological knowledge that exists in the labeled data which, even though not used in the classification process, serves as a guide in decision making to define which classes should be subdivided or attached.

Furthermore, unsupervised classification interactions, the class or classes selected to be subdivided from the method of the present invention, undergo a transformation process using the Robust Scaler method. Specifically, as is already known, the Robust Scaler method is a pre-processing technique used in machine learning to scale features (or variables) of a data set. It is especially useful when the data contains outliers, which are values that deviate significantly from most other data points. The goal of Robust Scaler is to reduce the impact of feature scale outliers, making data more suitable for scale-sensitive machine learning algorithms such as distance-based methods (e.g. k-NN) and algorithms involving numerical optimization (e.g. linear regression). Robust Scaler uses the median and interquartile range (IQR) to scale data. The idea is that the median is less sensitive to outliers than the mean, which helps minimize the effect of extreme points during scaling.

### STATE OF THE ART

In the state of the art, there are supervised machine learning techniques in electro-facies classification tasks.

The patent document US2022146705 A, for example, describes that the facies of a formation are classified based on data characterization properties of a portion of the formation as a function of depth, wherein the number of facies is determined automatically from unsupervised manner without human input. In one embodiment, a layer-based methodology is provided that performs facies classification based on layer-based properties that are determined from well logging data obtained from a plurality of different well logging tools. In another embodiment, a depth-based methodology is provided that performs facies classification based on well logging data obtained depth by depth from a plurality of different well logging tools. The number of facies can be determined automatically without human input, for example, using the Bayesian Information Criterion or a method that determines the optimal number of clusters based on the repeatability of the clustering results. In embodiments, facies classification may be performed using the Gaussian Mixture Model (GMM) method.

The document WO 22043051 A1 describes a method for determining an interpretation of electro-facies from measurements relating to at least one segment of at least one well passing through an underground formation. The method comprises applying a plurality of supervised or unsupervised classification methods to measurements for the purpose of determining training information. Subsequently, a plurality of supervised classification methods is applied to the measurements, the classification methods having been trained using the training information. Then, an ensemble classification method is applied to the results of the plurality of supervised classification methods to determine the electro-facies interpretation of the measurements.

The document US 2015/0241591 A1 discloses embodiments of systems, computer-implemented methods and non-transitory computer-readable medium with one or more computer programs stored therein provided to enhance well image analysis associated with a hydrocarbon reservoir. A neural network mapping process can first be performed, responsive to open hole logging data and core data, to thus generate a material type schema. Then, an image-based petrophysical analysis process can distribute and calibrate well image data, responsive to core data and material type Consequently, an approximate material type and approximate grain size can be produced for each well image reading. Open hole log data, core data, material type schema, and approximate material types and grain sizes, for example, can be displayed to increase consistency in categorizing subsurface material associated with hydrocarbon wells by material type and improve the interpretation of texture, fabric, and features of subsurface material to predict the composition of subsurface hydrocarbon reservoir material.

The document “A new tool for electro-facies analysis: multi-resolution graph-based clustering” (article by Shin-Ju Ye and Philippe Rabiller, published on Jun. 4, 2000, at the 1^{st} Annual Logging Symposium, Dallas, Texas), describes that log facies analysis is important for reservoir characterization, but is hampered mainly by the problem of “dimensionality”: log space is not equivalent to geological space, and two points that are close to each other in the log space may not always be similar geologically. A classic approach to facies analysis, automatic clustering, requires an estimate of the number of clusters, with the results being very sensitive to this parameter. If the clustering is tightly constrained, with few clusters, the analyst may find that, because of the “dimensionality” problem, the resulting clusters cannot be easily used for facies analysis. If the logging data is relatively unconstrained, the analyst will be faced with the difficult task of linking each cluster to a geological descriptor. Field experience shows that a two-step methodology provides a viable solution. First, a large number of clusters are chosen for automatic clustering. Second, small clusters are manually mixed into electro-facies that are assigned geological characteristics. Even with good visualization tools, performing this task manually in a high-dimensional space (>3) is still difficult, slow, somewhat subjective, and requires skill or experience that is not always readily available. This paper proposes a new method for electro-facies analysis, Multi-Resolution Graph-Based Clustering (MRGC), which solves the dimensionality problem and obtains valuable information about geological facies from the structure of the data itself. MRGC offers all the advantages while eliminating most of the disadvantages of the two-step method. MRGC is a multidimensional point pattern recognition method based on non-parametric representation of K-nearest neighbor and graphical data. The underlying structure of the data is analyzed, and groups of natural data are formed, that can have very different densities, sizes, shapes and relative separations. MRGC automatically determines the optimal number of clusters but allows the geologist to control the level of detail actually needed to define the electro-facies. This new electro-facie analysis tool has been tested under real-world conditions using conventional logging and NMR T2 distributions, and the results of such studies are shown in the paper. Compared to the existing two-step tool, MRGC was found to make the job much faster and easier, as well as more straightforward and intuitive.

The present invention differs from the state of the art because in the method it is possible to subdivide the profile data into different unsupervised classes and intuitively based on rock data interactively information, specifically, the method of the present invention seeks to represent geological knowledge that exists in the labeled data that, even though it is not used in the classification process, serves as a guide in decision making to define which classes should be subdivided or attached.

### BRIEF DESCRIPTION OF THE INVENTION

The present invention relates to a computer-implemented method for multi-scale unsupervised classification of electro-facies, comprising: (a) entering profile data; (b) transforming the profile data using the robust scaler method; (c) subdividing the profile data into unsupervised classes; (d) comparing the result of the unsupervised classes with the rock data, which are irregularly distributed throughout the well; (e) the user selects which unsupervised classes will be subjected to a new unsupervised classification, optionally one or more classes; (f) transforming only the data that comprises the selected class or classes; and (g) performing unsupervised classification, wherein the clusters will have a non-linear character. In which the profile data generates the number of classes defined by the user. In step (d) each unsupervised class is optionally analyzed with rock data that does not enter the classification. In step (e) the choice of the number of unsupervised classes that will be generated is made by the user based on geological knowledge or what the rock data indicates. Since all unsupervised classification interactions, the data comprising the class or classes selected to be subdivided are transformed in step (f) by the Robust Scaler method. In step (g) the clusters will no longer be fully configured according to the distance between the original points, but rather due to the perspective of the user when assigning geological knowledge to unsupervised classes. Furthermore, in step (g), eventually, the classifications will not be able to individualize the rock classes and new interactions (e), (f) and (g) may be carried out. Furthermore, in step (g) the unsupervised classification is determined by the user creating the model.

Furthermore, the present invention relates to a computer-readable non-transitory storage medium comprising instructions stored therein, wherein the instructions, when read by a computer, cause the computer to perform the steps of the computer-implemented method for multi-scale unsupervised classification of electro-facies.

### BRIEF DESCRIPTION OF THE FIGURES

In order to complement the present description and obtain a better understanding of the characteristics of the present invention, and in accordance with a preferred embodiment thereof, in annex, a set of figures is submitted, where in an exemplified, although not limiting, manner, represents the preferred embodiment.

FIG. 1 shows a scheme of how the method of the present invention is applied on several different scales, from macro characteristics, refining to more specific characteristics, with the aid of rock data, according to a preferred embodiment of the present invention.

FIG. 2 is a representation of an example of the present invention in which, from the profile data (a) the unsupervised method subdivides the data into two unsupervised classes (b), resulting in an electro-facies curve (w). Each unsupervised class can be analyzed with rock data (d) which, although not entered into the classification process, can be used to help defining the next steps, according to a preferred embodiment of the present invention.

FIG. 3 is a representation of an example of the present invention in which, from the unsupervised classes created in the first iteration (FIG. 2) (a), it is possible to select which ones will be subjected to a new round of unsupervised classification (b), generating new unsupervised classes (c) in an attempt to individualize the classes of the rock data (d), according to a preferred embodiment of the present invention.

FIG. 4 is a representation of the present invention in which, using the result of an unsupervised classification (a), when selecting two classes (b) that are not necessarily neighbors in the space of mathematical characteristics of the variables and if applying a transformation to the data (c) so that they are in a known distribution, for example, mean zero and standard deviation one, completely distorts the original distances that these samples have with other samples that are not part of these classes, according to a preferred embodiment of the present invention.

FIG. 5 is a representation of the present invention in which, from the unsupervised classes generated in the previous interaction, two classes are selected for a new classification, the blue class and the yellow class, wherein these samples are subjected to the unsupervised method of the present invention and three new classes are generated, improving the segmentation of rock data representativeness into unsupervised classes, according to a preferred embodiment of the present invention.

FIG. 6 is a representation of the present invention in which, from the unsupervised classes (a), it is possible to select classes that will be attached to a single class (b), reducing the number of unsupervised classes (c) and creating a new proportion of rock data per unsupervised class (d), in accordance with a preferred embodiment of the present invention.

FIG. 7 is a representation of the present invention in which, from the unsupervised model, the generated labels are used to train a supervised model (c) which will subsequently be fed with new data (d) which will be mapped according to model (b) and then classified (e), so that the supervised model generates class probability curves that were reinterpreted as class membership curves (f), according to a preferred embodiment of the present invention.

### DETAILED DESCRIPTION OF THE INVENTION

Specifically, the dynamics of the methodology of the present invention was created so that the result is constructed little by little and intuitively, just like the process of analyzing an outcrop or rock core. Therefore, electrical profile data is initially used to identify the most relevant and easily separable macro characteristics, such as reservoir and non-reservoir rock. The ability to interactively subdivide or attach classes gives the freedom to test scenarios and validate concepts quickly.

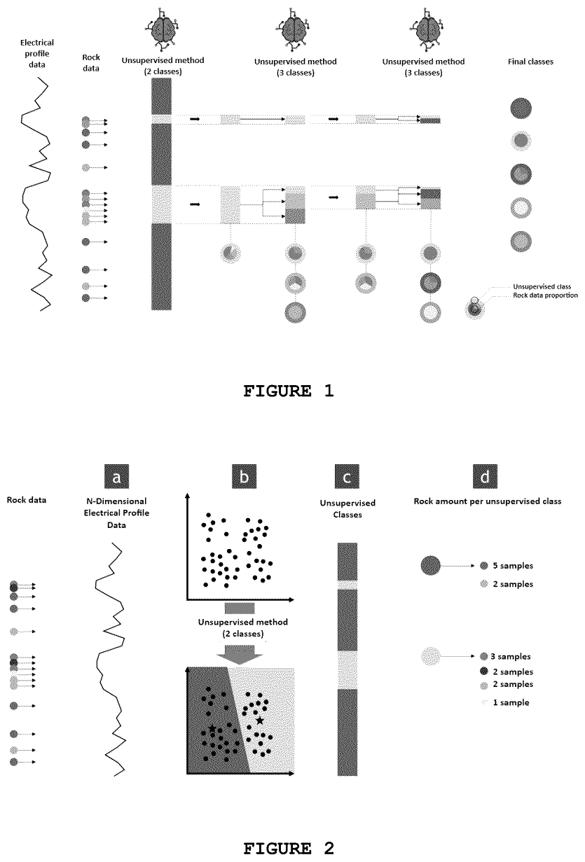

In this sense, it is quite intuitive to start from a macro analysis and gradually identify more specific characteristics based on the geological knowledge of a specialist or what the rock data indicates. Therefore, the methodology allows countless interactions to be carried out until achieving the desired result, as seen in FIG. 1.

It should be noted that all unsupervised classification interactions, the class or classes selected to be subdivided go through a transformation process using the Robust Scaler method, as observed in FIG. 4. This process has an important consequence on the result of the method model.

In general, in unsupervised methods, usually measuring the distance between points is one of the essences of how the methods work. When two or more classes are selected, which are not necessarily in the same context, and this data is transformed, completely modifying the distances between the samples, a non-linear character is inserted into the model. The aim of this way of dealing with data, exclusive to the proposed method, attempts to overcome the impasse mentioned by Ye [1], wherein he states that the distance of mathematical characteristics between classes of electro-facies does not always agree with the distance of geological significance between the two classes. In other words, two rocks that are geologically very similar are not necessarily close in the of mathematical space characteristics and may even have rocks that are very different and closer in this space.

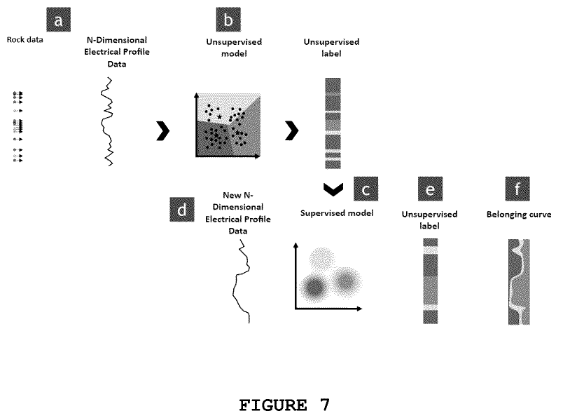

Furthermore, in addition to the possibility of subdividing unsupervised classes into new classes, in the method of the present invention it is also possible to attach unsupervised classes into a single class (b), as seen in FIG. 6. This process was added to the method because the subdivision of classes does not always generate the expected result.

In this way, the possibility of joining classes not only allows classification errors to be restored, but also gives a non-linear character to the clusters, as they will no longer be fully configured according to the distance between the points, but rather due to the user (interpreter) perspective when assigning geological knowledge to unsupervised classes. Therefore, the classifications will not always be able to individualize the rock classes (d), as seen in FIG. 6, as these may not be separable using only the model variables or, simply, not be coherent with the available profile data or desired result.

Completion of the unsupervised classification method is determined by the user creating the model. This must end interactions when, based on the information provided by different forms of data visualization, the classes converge to geological meanings or electrical profiles that the user judges according to the objective of the classification. This increases the subjective nature of the methodology, making it flexible to the bias of the user.

After successive interactions subdividing and attaching unsupervised classes until finding the final model, it is necessary to save the solution so that it can be used later on other data. If the model were the result of a single unsupervised interaction, it would be enough to save the information about the centroids of each unsupervised cluster and, later with the new data, assign each of the centroids according to the distance between the points. However, as seen previously, successive unsupervised interactions are carried out and, in each one, the sample space is transformed by the Robust Scaler method and, in addition, it is possible to use more than one type of unsupervised method, where the creation criteria of clusters differ between them. For these reasons, the solution of saving the centroids of each cluster is discarded.

To solve this problem, it is proposed to use a supervised method based on the labels generated by the unsupervised model to create a model that will then be used on new data (d), in FIG. 7, for example. The method used is the Random Forest classifier, based on decision trees (c), in FIG. 7. Consequently, the performance of the supervised method will depend on the complexity of the model generated in the unsupervised stage.

An advantage of this type of approach is the amount of labeled data (unsupervised classes) compared to the existing rock data (a), in FIG. 7, which are few and unbalanced. Therefore, the characteristics of unsupervised classes are more likely to be well mapped by the supervised method (c) in FIG. 7, for example. The supervised method used (Random Forest classifier), in addition to the deterministic classification (e), in FIG. 7, also generates class probability curves (f), in FIG. 7, which are calculated from the several predictions made by the method estimators based on decision trees. When talking about unsupervised clustering methods, the concept of centroid is widely used and is usually the point at which the most relevant characteristics of that class are found on average. Therefore, the further away from the centroid, the less similar the sample is to it.

Consequently, the peripheries of the clusters will have ambiguous characteristics and samples in these regions will be classified with greater uncertainty. Based on this understanding, the concept of belonging to the cluster was explored, where samples with high probability in a given class mean that they have characteristics close to the centroid, while those with low probability will be less characterized by this (f) in FIG. 7, for example.

Finally, in another embodiment of the present invention, a non-transitory computer-readable medium is provided. The medium may be, for example, a memory, a flash memory, a hard disk, a compact disk, or any other device capable of storing computer instructions. When the readable medium of the present embodiment is read by a computer, the computer is enabled to perform the method for multi-scale unsupervised classification of electro-facies.

### Example of Implementation/Tests/Results

In FIG. 2, an example of the computer-implemented method of the present invention can be seen, wherein the profile data (a) is initially subjected to the unsupervised classification algorithm, which generates the number of classes defined by the user, in this case two unsupervised classes (b) and (c). The result can be compared with the rock data (d) from lateral samples, which are irregularly distributed throughout the borehole. In the example, the blue unsupervised class can individualize a rock class very well, while the yellow unsupervised class covers four different rock classes.

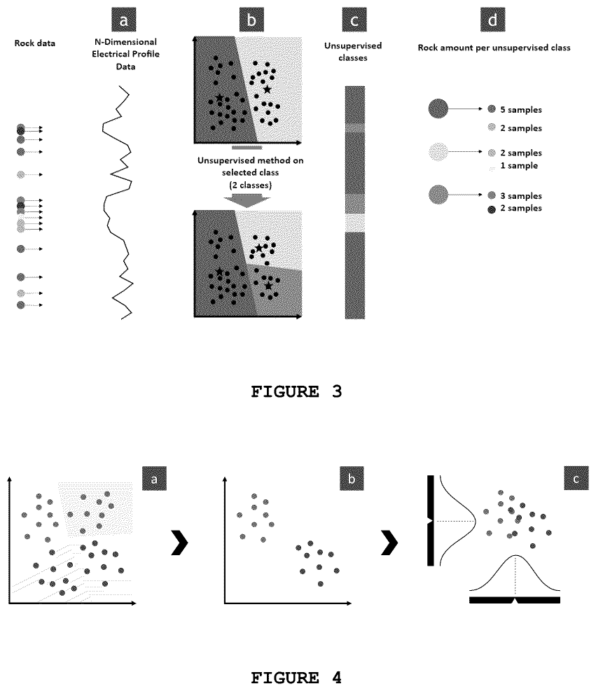

In this preferred embodiment of the present invention, represented in FIG. 2, based on the analysis between the unsupervised classes and the rock data classes, the most interesting decision is to subdivide the yellow unsupervised class in an attempt to individualize the rock data classes. In this subsequent step, the user has control over selecting which unsupervised classes will be submitted to the unsupervised classification method, which may be one or more classes. As previously in FIG. 2, the choice of the number of unsupervised classes that will be generated is also a user choice and must be based on geological knowledge or what the rock data indicates. In the example seen in FIG. 3, the yellow unsupervised class will be subdivided into 2 new classes, totaling 3 unsupervised classes (b) and (c).

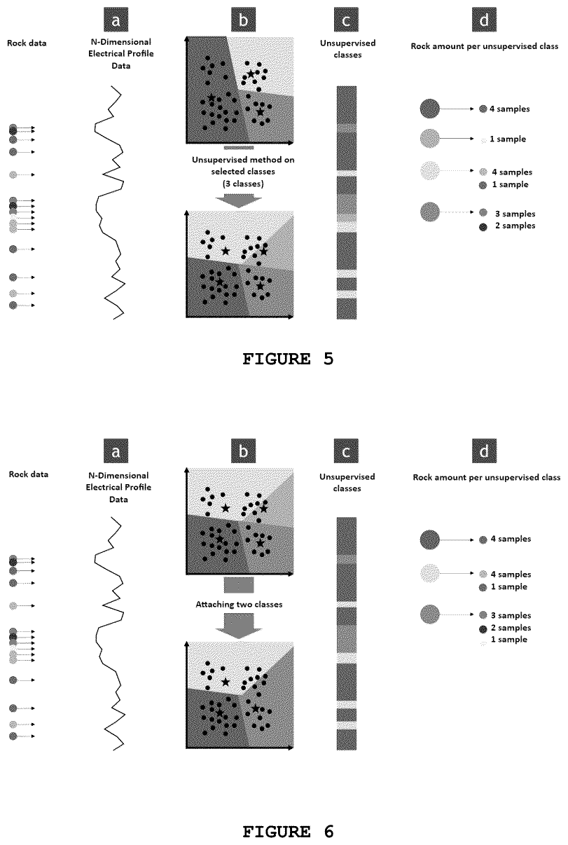

In said preferred embodiment of the present invention, when selecting two classes to perform a new unsupervised classification, the Euclidean distances between the selected samples are distorted. The consequence is that assigning unsupervised classes based on the distance of the samples to the centroids will no longer make sense. In the example seen in FIG. 5, two unsupervised classes are selected (blue and yellow). The profile samples (a) represented in FIG. 5, belonging to these two classes will then be transformed, using the Robust Scaler method, and then submitted to the unsupervised classification method (b), thus generating three new classes (c) with representativeness of better discriminated rock data (d).

### BIBLIOGRAPHIC REFERENCES

Ye, S. J. and Rabiller, P. (2000) A New Tool for Electro-Facies Analysis: Multi-Resolution Graph-Based Clustering. SPWLA 41^{st} Annual Logging Symposium, Society of Petrophysicists and Well-Log Analysts.
Serra & Abbot (1980)

1. A computer-implemented method for multi-scale unsupervised classification of electro-facies comprising the following steps:
(a) inserting profile data;
(b) transforming the profile data using the robust scaler method;
(c) subdividing the profile data into unsupervised classes;
(d) comparing the result of the unsupervised classes with the rock data, which are irregularly distributed throughout the well;
(e) the user selects which unsupervised classes will be subjected to a new unsupervised classification, optionally one or more classes; and
(f) transforming only the data that comprises the selected class or classes;
(g) performing unsupervised classification, wherein the clusters will have a non-linear character.

2. The method according to claim 1, wherein the profile data generates the number of classes defined by the user.

3. The method according to claim 1, wherein in step (b) each unsupervised class, optionally, is analyzed with rock data that does not enter the classification.

4. The method according to claim 2, wherein in step (e) the choice of number of unsupervised classes that will be generated is made by the user based on geological knowledge or what the rock data indicates.

5. The method according to claim 1, wherein all unsupervised classification interactions, the class or classes selected to be subdivided undergo a transformation process using a Robust Scaler method.

6. The method according to claim 1, wherein there is the possibility of subdividing unsupervised classes into new classes and attaching unsupervised classes to a single class.

7. The method according to claim 1, wherein in step (g) the clusters will no longer be fully configured according to the distance between the points, but rather due to the perspective of the user when attributing geological knowledge to the unsupervised classes.

8. The method according to claim 1, wherein in step (g), eventually, the classifications will not be able to individualize the rock classes and new interactions of steps (e), (f) and (g).

9. The method according to claim 1, wherein in step (g) the unsupervised classification is determined by the user who is creating the model.

10. A computer-readable non-transitory storage medium comprising instructions stored therein wherein the instructions, when read by a computer, cause the computer to perform the steps of the method as defined in claim 1.
